(19) United States
(12) Patent Application Publication
(10) Pub. No.: US 2026/0366184 A1
(43) Pub. Date: Nov. 19, 2026

(54) GREEN HYDROGEN GENERATION APPARATUS

(71) Applicants: NZW INC., Seoul (KR); KOLON GLOBAL CORPORATION, Gyeonggi-do (KR)

(72) Inventors: DAE YEOL CHEONG, Seoul (KR); HO JOON LEE, Seoul (KR); JIN SU CHOI, Seoul (KR)

(21) Appl. No.: 19/144,085

(22) PCT Filed: Nov. 10, 2023
(86) PCT No.: PCT/KR2023/018070

§ 371 Date: Jun. 27, 2025

(30) Foreign Application Priority Data
Dec. 29, 2022 (KR) 10-2022-0188909

(51) Int. Cl. C25B 15/08; B01D 53/047; B01D 53/22; C02F 1/00; C02F 1/24; C02F 1/40; C02F 1/44; C02F 1/66; C02F 3/00; C02F 3/28; C02F 9/00; C02F 101/32; C02F 103/06; C02F 103/32; C25B 1/04; C25B 9/17

(52) U.S. Cl. CPC C25B 15/085; B01D 53/0476; B01D 53/229; C02F 9/00; C25B 9/17; B01D 2256/16; B01D 2258/05; C02F 1/004; C02F 1/24; C02F 1/40; C02F 1/44; C02F 1/66; C02F 3/005; C02F 3/2866; C02F 2101/32; C02F 2103/06; C02F 2103/32; C25B 1/04

(57) ABSTRACT

A green hydrogen generation apparatus includes a pre-treatment unit that pre-treats and pre-ferments source water composed of food waste and leachate, an MEC unit that electrolyzes microorganisms in the source water that is pre-treated and pre-fermented in the pre-treatment unit, and a post-treatment unit that purifies and compresses hydrogen gas generated through electrolysis in the MEC unit. The MEC unit lowers a concentration of organic components in the source water by increasing reaction time or retention time or increases a concentration of input organic matter to increase a generation amount of hydrogen gas.

### BACKGROUND

### 1. Technical Field

The present disclosure relates to a green hydrogen generation apparatus that generates and processes green hydrogen from food waste and leachate.

### 2. Background Art

13,701 tons of food waste is generated per day, and the amount generated per person per day is 0.28 kg, which accounts for 27.7% of the total 50,906 tons of daily household waste, accounting for a significant portion of the total household waste.

In addition, when looking at the physical properties of food waste, the food waste is composed of vegetables, fruits, grains, fish, meat, etc., and usually has a moisture content of 80 to 85%, so it has a total solid (TS) content of 15% or more and a high decomposition rate of 80% or more compared to organic wastes such as livestock waste, manure, and sewage sludge.

Most of the food waste is being processed through feed production or composting, but due to problems such as deterioration in the quality of feed or compost that is final byproducts, and difficulty in securing demand, an anaerobic digestion treatment apparatus for food waste is being adopted as part of the low-carbon green policy, which generate biogas through anaerobic digestion of food waste and use the generated biogas as an alternative energy source for liquefied natural gas (LNG).

However, the conventional anaerobic digestion treatment apparatuses for food waste, which are currently installed and operating in South Korea, have many problems due to problems such as not being suitable for the properties of domestic food waste due to the process of directly introducing foreign technology, inappropriate pre-treatment facilities, decreased efficiency of an anaerobic digestion tank due to excessive input of high-concentration solids into a single-phase anaerobic digestion tank, and accumulation of organic acids in the anaerobic digestion tank due to unstable stirring of the anaerobic digestion tank.

In particular, the conventional anaerobic digestion treatment apparatuses for food waste mostly use simple shredding and sorting devices for pre-treatment of anaerobic digestion facilities, so the removal rate of foreign substances is kept low, and the retention time is relatively short, within 30 days, with little consideration for particle size.

Therefore, the conventional anaerobic digestion treatment apparatuses for food waste have problems in that normal operation of the anaerobic digestion tank becomes impossible due to the accumulation of foreign substances in the anaerobic digestion tank, the increase in dead space of the anaerobic digestion tank, and the increase in organic acids, etc. As a result, the amount of food waste processed gradually decreases, and ultimately, the input of food waste into the digestion tank becomes impossible.

### SUMMARY

Therefore, the present disclosure has been made to solve the above problems, and provides an integrated green hydrogen generation apparatus that pre-treats and pre-ferments food waste or leachate and converts the food waste or the leachate into an MEC-tailored organic substrate suitable for hydrogen generation in a MEC, thereby enabling the generation of green hydrogen in an MEC reactor.

However, technical problems of the present disclosure are not limited to the above-mentioned technical problems. That is, other technical problems that are not mentioned may be obviously understood by those skilled in the art to which the present disclosure pertains from the following description.

In one general aspect, a green hydrogen generation apparatus includes: a pre-treatment unit that pre-treats and pre-ferments source water composed of food waste and leachate; an MEC unit that electrolyzes microorganisms in the source water that is pre-treated and pre-fermented in the pre-treatment unit; and a post-treatment unit that purifies and compresses hydrogen gas generated through electrolysis in the MEC unit, in which the MEC unit lowers a concentration of organic components in the source water by increasing reaction time or retention time or increases a concentration of input organic matter to increase a generation amount of hydrogen gas.

The pre-treatment unit may include: a three-phase separator that separates oil and impurities from the food waste and the leachate; a pressurized flotation tank that separates and removes pollutants in treated water transferred from the three-phase separator by floating the pollutants; a pre-fermentation tank that generates organic acids through anaerobic acid fermentation by utilizing organic matter separated from the treated water transferred from the pressurized flotation tank; an effluent filtration tank that removes and reduces floating total solids in the treated water transferred from the pre-fermentation tank; and a conditioning tank that adjusts and buffers a pH in the treated water transferred from the effluent filtration tank.

The MEC unit may include: an MEC tank that generates hydrogen through microbial electrolysis using a pre-treated soluble organic matter; a storage tank that provides a flow rate from an external reaction tank to the MEC tank for supplying a circulating flow rate to the MEC tank; and a trap tank that separates a gas and solution coming out through a negative electrode of the MEC tank and returns the solution back to the storage tank.

The three-phase separator may perform oil separation or solid-liquid separation by inputting a chemical cohesive agents set according to properties of the source water.

The pressurized flotation tank may control a concentration of a particulate matter in the treated water transferred from the three-phase separator through a pressurized flotation process.

The pre-fermentation tank may perform acid fermentation for the set retention time while adjusting the pH of the treated water.

The effluent filtration tank may be provided with a cross-flow cross-filtration membrane to maintain the flow rate of the treated water at a set level.

The conditioning tank may maintain the pH of the treated water at 6.0 to 7.5.

The post-treatment unit may purify, compress, and liquefy the hydrogen gas generated through the MEC unit and transmit the hydrogen gas to a hydrogen charging station.

The post-treatment unit may be connected to a wastewater treatment facility to remove organic components and nutrients generated through the MEC unit.

According to an embodiment of the present disclosure, a pre-treatment and pre-fermentation process may remove oil, coarse particles, impurities, etc., in source water through physicochemical treatment of food waste and leachate, and convert food waste and leachate into organic acids suitable for use in an MEC unit.

In addition, according to an embodiment of the present disclosure, an MEC unit generating green hydrogen is a two-chamber MEC unit using a microbial electrolysis method, unlike the existing hydrogen generation process, and may generate the green hydrogen by introducing MEC-tailored pre-fermentation inflow source water into a reactor inflow stage.

In addition, according to one embodiment of the present disclosure, it is possible to secure energy supply facilities through the treatment of high-concentration organic wastewater such as food waste and leachate, and connection with green hydrogen generation and charging facilities by an integrated design configuration.

Effects which can be achieved by the present disclosure are not limited to the above-described effects. That is, other objects that are not described may be obviously understood by those skilled in the art to which the present disclosure pertains from the following description.

### BRIEF DESCRIPTION OF THE DRAWINGS

The following drawings attached to this specification illustrate preferred embodiments of the present disclosure, and together with the detailed description of the invention described below, serve to further understand the technical idea of the present disclosure, so the present disclosure should not be interpreted as being limited to matters described only in such drawings:

FIG. 1 is an overall block diagram of a green hydrogen generation apparatus according to an embodiment of the present disclosure;

FIG. 2 is a block diagram of components of a pre-treatment unit;

FIG. 3 is a block diagram of components of an MEC unit;

FIG. 4 is a flowchart of the overall operation of the green hydrogen generation apparatus; and

FIG. 5 is an overall conceptual diagram of the green hydrogen generation apparatus.

### DETAILED DESCRIPTION

Hereinafter, embodiments of the present disclosure will be described in detail with reference to the accompanying drawings so that those skilled in the art may easily practice the present disclosure. However, description of the present disclosure is only an embodiment for structural or functional description, and therefore the scope of the present disclosure should not be construed as limited to embodiments described in the text. That is, since the embodiments may be variously modified and may have various forms, the scope of the present disclosure should be construed as including equivalents capable of realizing the technical idea. In addition, a specific embodiment is not construed as including all the objects or effects presented in the present disclosure or only the effects, and therefore the scope of the present disclosure should not be understood as being limited thereto.

The meaning of the terms described in the present application should be understood as follows.

Terms such as “first” and “second” are intended to distinguish one component from another component, and the scope of the present disclosure should not be limited by these terms. For example, a first component may be named a second component and the second component may also be similarly named the first component. It is to be understood that when one element is referred to as being “connected to” another element, it may be connected directly to or coupled directly to another element or be connected to another element, having the other element intervening therebetween. On the other hand, it is to be understood that when one element is referred to as being “connected directly to” another element, it may be connected to or coupled to another element without the other element intervening therebetween. Meanwhile, other expressions describing a relationship between components, that is, “between”, “directly between”, “neighboring to”, “directly neighboring to” and the like, should be similarly interpreted.

It should be understood that the singular expression include the plural expression unless the context clearly indicates otherwise, and it will be further understood that the terms “comprises” or “have” used in this specification, specify the presence of stated features, steps, operations, components, parts, or a combination thereof, but do not preclude the presence or addition of one or more other features, numerals, steps, operations, components, parts, or a combination thereof.

Unless defined otherwise, all the terms used herein including technical and scientific terms have the same meaning as meanings generally understood by those skilled in the art to which the present disclosure pertains. It should be understood that the terms defined by the dictionary are identical with the meanings within the context of the related art, and they should not be ideally or excessively formally defined unless the context clearly dictates otherwise in the present disclosure.

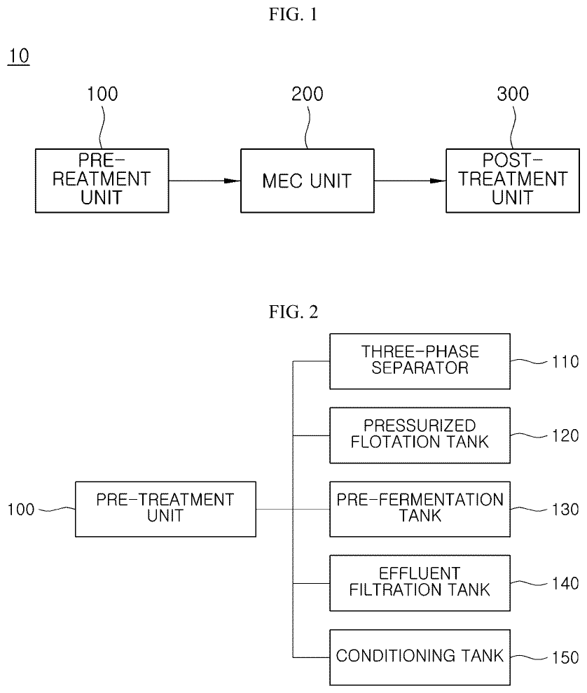

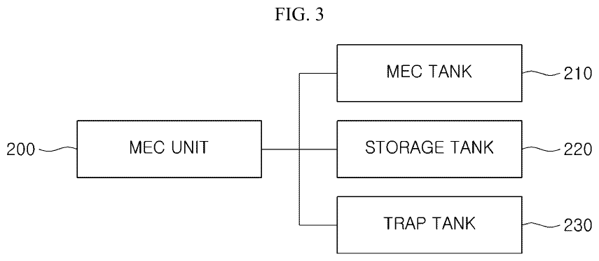

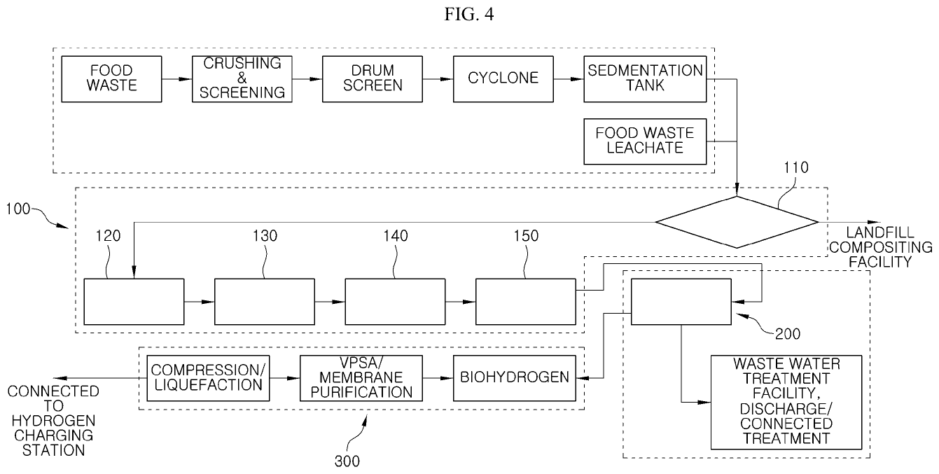

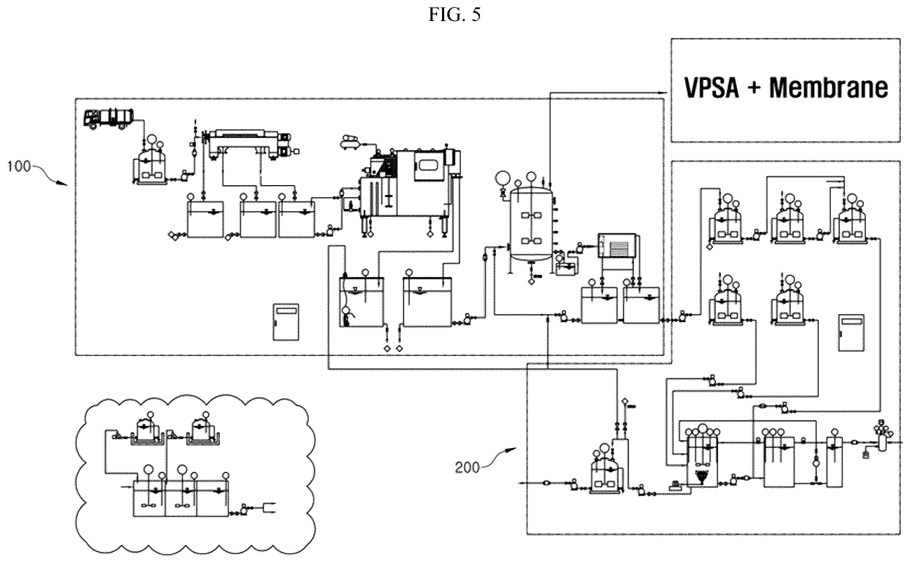

FIG. 1 is an overall block diagram of a green hydrogen generation apparatus according to an embodiment of the present disclosure, FIG. 2 is a block diagram of components of a pre-treatment unit, FIG. 3 is a block diagram of components of an MEC unit, FIG. 4 is a flowchart of the overall operation of the green hydrogen generation apparatus, and FIG. 5 is an overall conceptual diagram of the green hydrogen generation apparatus.

As illustrated in FIGS. 1 to 5, the present disclosure may include a pre-treatment unit 100, an MEC unit 200, and a post-treatment unit 300.

The pre-treatment unit 100 may pre-treat and pre-ferment source water composed of food waste and leachate.

The pre-treatment unit 100 may include a three-phase separator 110, a pressurized flotation tank 120, a pre-fermentation tank 130, an effluent filtration tank 140, and a conditioning tank 150.

The three-phase separator 110 may separate oil and impurities from food waste and leachate. The installed flow rate of the three-phase separator 110 is 0.1 to 0.2 m³/hr or 1 to 2 m³/hr, and an oil removal rate of treated water after separation may be 95% or higher. Depending on the properties of the source water, a chemical cohesive agents set may be added to perform oil separation or solid-liquid separation.

The pressurized flotation tank 120 may float pollutants in the treated water transferred from the three-phase separator 110 and separate and remove the pollutants. The pressurized flotation tank 120 may control a concentration of particulate matter in the treated water transferred from the three-phase separator 110 through a pressurized flotation process.

Specifically, an installed flow rate of the pressurized flotation tank 120 allows a retention time to be 0.5 to 1 hr, a removal rate of the floating total solid may be 80% or higher, and a liquid phase, which is source water separated from the food waste and the leachate through the three-phase separator 110, may control the particulate matter through the pressurized flotation process. Depending on the properties of the source water, the concentration of the particulate matter may be reduced by adjusting the pH and inputting cohesive agents as an auxiliary.

The cohesive agents are chemicals added to a liquid to make several solid particles suspended in the liquid gather together to form slightly larger lumps. When cohesion occurs, a sedimentation speed of particles in the liquid generally increases, so this phenomenon may be used to promote sedimentation.

The cohesive agents are also called sedimentation accelerators or sedimentation agents, and may be largely divided into two types based on chemical composition. The first is inorganic electrolytes, such as slaked lime (calcium hydroxide), alum, aluminum chloride, iron (III), and iron (II) sulfate. These may cause coagulation by making a surface potential of particles in a liquid approach almost 0 and eliminating an electrical repulsion between particles that exist until then.

The second is organic polymer compounds, such as starch, polyacrylamide, and its derivatives. The starch causes coagulation by the increase/decrease effect of colloids, while polyacrylamide and its derivatives are thought to attract particles to each other by a bridging effect of molecules adsorbed to the particles. In most cases, the effect is greater when the two cohesive agents are used together.

The pre-fermentation tank 130 may generate organic acids through anaerobic acid fermentation by utilizing organic matter separated from the treated water transferred from the pressurized flotation tank 120. The pre-fermentation tank 130 may perform acid fermentation for the set retention time while adjusting the pH of the treated water.

Specifically, the pre-fermentation tank 130 is equipped with a low-speed stirrer (not illustrated), and may have a hydraulic retention time of 0.5 to 2 days at an installed flow rate and a minimum reactor capacity of 1 ton. After removing oil, coarse particles, and particulate matter from food waste and leachate, the food waste and the leachate are put into the acid fermentation tank, the pH is adjusted, and the acid fermentation may be performed through a set retention time. That is, the corresponding process in the pre-fermentation tank 130 is an acid fermentation process with acetate and ethanol as the main components of the final by-products, and the acid fermentation process may enhance a purification effect of the treated water in the reaction tank by incorporating a sedimentation process within the reaction tank.

The purification is to separate fine particles floating in the solution from the solution by some operation, and general purification methods include natural sedimentation, adsorbent (cohesive agent) addition, filtration, etc., and these methods may be used alone or in combination. Even in the wet reprocessing method, the purification is an important operation to remove insoluble residual particles from the fuel solution before the extraction operation.

The effluent filtration tank 140 may remove and reduce the floating total solids in the treated water transferred from the pre-fermentation tank 130. The effluent filtration tank 140 may be provided with a cross-flow cross-filtration membrane to maintain the flow rate of the treated water at a set level.

Specifically, the effluent filtration tank 140 is in charge of the filtration process that finally controls the particulate matter in order to put the treated water fermented in the pre-fermentation tank 130 into the MEC unit 200. To this end, by applying the cross-flow cross-filtration membrane (not illustrated), unlike the chemical cleaning and backwashing processes of the existing conventional membrane separation process, the generated water flow rate may be consistently maintained and lower filtration media exchange may be required than the existing membrane separation process.

The conditioning tank 150 may adjust and buffer the pH in the treated water transferred from the effluent filtration tank 140. The conditioning tank 150 may maintain the pH of the treated water at 6.0 to 7.5. Specifically, the conditioning tank 150 is a reaction tank equipped with a rapid and slow stirrer (not illustrated) to adjust the properties of the final source water, so that the pH is maintained at a preset condition of 6.0 to 7.5 before being introduced into the final MEC unit 200, thereby increasing the operational stability and efficiency of the MEC unit 200.

The MEC unit 200 may electrolyze microorganisms in the source water that has been pre-treated and pre-fermented in the pre-treatment unit 100. Here, the MEC is an abbreviation for a microbial electrolysis cell, and means a microbial electrolysis cell. Hereinafter, the microbial electrolysis cell will be described using the abbreviation MEC.

The MEC unit 200 may lower a concentration of organic components in the source water by increasing reaction time or retention time or increase a concentration of input organic matter, thereby increasing the generation amount of hydrogen gas.

The MEC unit 200 may include an MEC tank 210, a storage tank 220, and a trap tank 230.

The MEC tank 210 may generate hydrogen through microbial electrolysis using a pre-treated soluble organic matter. The installed flow rate of the MEC tank 210 is 62.5 L per tank, with a module composed of four tanks totaling 250 L. Four modules can be installed in the pilot facility.

The storage tank 220 may provide a flow rate from an external reaction tank to the MEC tank 210 in order to supply a circulating flow rate to the MEC tank 210. The installed flow rate of the storage tank 220 may be matched 1:1 with the MEC tank 210, and the low-speed stirrer (not illustrated) with the performance of 30 to 60 rpm (the low-speed stirrer is about 60 rpm) and a circulating flow rate pump (not illustrated) with the performance of 2.4 to 24 L/hr may be equipped inside the storage tank 220.

The trap tank 230 may separate the gas and solution coming out through the negative electrode of the MEC tank 210 and return the solution back to the storage tank 220. The installed flow rate of the trap tank 230 may be 1 to 5 L per tank.

The post-treatment unit 300 may purify and compress the hydrogen gas generated by electrolysis in the MEC unit 200.

The post-treatment unit 300 may purify, compress, and liquefy the hydrogen gas generated through the MEC unit 200 and transmit the hydrogen gas to a hydrogen charging station.

Specifically, the post-treatment unit 300 may input green hydrogen itself, which is a biogas generated through the MEC unit 200 and has a purity of 90% or higher, into a generator, etc., to obtain energy.

In addition, the post-treatment unit 300 may purify biohydrogen into high-purity hydrogen through a VPSA/membrane composite process that satisfies the ISO standard of 99.995% or higher and secures a purification recovery rate of 90% or higher.

In addition, the post-treatment unit 300 is a place where an additional process is performed to process, compress, and liquefy the high-purity hydrogen generated through the MEC unit 200 into a form distributed in the market so that consumers may use the high-purity hydrogen directly. The solids of organic wastewater generated from the green hydrogen generation apparatus 10 may be processed by connecting the green hydrogen generation apparatus 10 to a composting facility, a biogasification facility, a landfill facility, etc.

In addition, the food waste may be liquefied/slurried to be the leachate, and the food waste and leachate generated may be homogenized in a storage tank and input to the pre-treatment unit 100.

It may be connected to a wastewater treatment facility to remove organic components and nutrients generated through the MEC unit 200.

The process of the green hydrogen generation apparatus according to one embodiment of the present disclosure is as follows.

Referring to FIG. 4, the food waste is crushed and screened and passed through a drum screen, a cyclone, and a sedimentation tank, respectively, to generate the food waste and the leachate as source water, and the source water may be transferred to the pre-treatment unit 100.

In the pre-treatment unit 100, as described above, the source water is processed through the three-phase separator 110, the pressurized flotation tank 120, the acid fermentation tank 130, the effluent filtration tank 140, and the conditioning tank 150 using various methods such as separation, compression, and sedimentation, and the resulting treated water may be transferred to the MEC unit 200.

The treated water transferred to the MEC unit 200 generates green hydrogen through a microbial electrolysis reaction and is transferred to the post-treatment unit 300. The green hydrogen, i.e., biohydrogen, transferred to the post-treatment unit 300 is purified through processes such as vacuum, pressure, swing, and adsorption (VPSA, Vacuum Pressure Swing Adsorption), and may be compressed and liquefied before being transferred to the connected hydrogen charging station.

In addition, the wastewater generated from the treated water transferred to the MEC unit 200 may be treated and discharged through the connected wastewater treatment facility.

According to an embodiment of the present disclosure, the pre-treatment and pre-fermentation process may remove oil, coarse particles, impurities, etc., in the source water through the physicochemical treatment of the food waste and the leachate, and convert the food waste and the leachate into the organic acids suitable for use in the MEC unit 200.

In addition, according to an embodiment of the present disclosure, unlike the existing hydrogen generation process, the MEC unit 200 producing the green hydrogen is a two-chamber MEC unit 200 with the MEC tank 210 and the trap tank 230 using a microbial electrolysis method, and may produce green hydrogen by introducing MEC-tailored pre-fermentation inflow source water into a reaction tank inflow stage.

In addition, according to one embodiment of the present disclosure, it is possible to secure the energy supply facilities through the treatment of the high-concentration organic wastewater such as the food waste and the leachate, and the connection with the green hydrogen generation and charging facilities by the integrated design configuration of the pre-treatment unit 100 and the MEC unit 200.

The detailed description of the preferred embodiments of the present disclosure disclosed above has been provided to enable those skilled in the art to implement and practice the present disclosure. Although the above has been described with reference to preferred embodiments of the present disclosure, it will be understood that those skilled in the art may variously modify and change the present disclosure without departing from the scope of the present disclosure. For example, those skilled in the art may utilize each component described in the above-described embodiments in a manner that combines the components. Accordingly, the present disclosure is not intended to be limited to the embodiments disclosed herein, but is intended to have the widest scope consistent with the principles and novel features disclosed herein. The present disclosure may be embodied in other specific forms without departing from the spirit and essential characteristics of the present disclosure. Therefore, the above-mentioned detailed description is to be interpreted as being illustrative rather than being restrictive in all aspects. The scope of the present disclosure is to be determined by reasonable interpretation of the claims, and all modifications within an equivalent range of the present disclosure fall in the scope of the present disclosure. The present disclosure is not intended to be limited to the embodiments disclosed herein, but is intended to have the widest scope consistent with the principles and novel features disclosed herein. Claims that do not have an explicitly referred relationship in the claims may be combined with each other to configure an embodiment or be included in new claims by amendment after application.

A green hydrogen generation apparatus according to one embodiment of the present disclosure has industrial applicability because the pre-treatment and pre-fermentation process may remove oil, coarse particles, impurities, etc., in the source water through the physicochemical treatment of the food waste and leachate, and convert the food waste and leachate into organic acids suitable for use in the MEC unit.

1. A green hydrogen generation apparatus, comprising:
a pre-treatment unit configured to pre-treat and pre-ferment source water comprised of food waste and leachate;
a microbial electrolysis cell (MEC) unit configured to electrolyze microorganisms in the source water that is pre-treated and pre-fermented in the pre-treatment unit; and
a post-treatment unit configured to purify and compress hydrogen gas generated through electrolysis in the MEC unit,
wherein the MEC unit is configured to lower a concentration of organic components in the source water by increasing reaction time or retention time or increase a concentration of input organic matter to increase a generation amount of hydrogen gas.

2. The green hydrogen generation apparatus of claim 1, wherein the pre-treatment unit includes:
a three-phase separator configured to separate oil and impurities from the food waste and the leachate;
a pressurized flotation tank configured to separate and remove pollutants in treated water transferred from the three-phase separator by floating the pollutants;
a pre-fermentation tank configured to generate organic acids through anaerobic acid fermentation by utilizing organic matter separated from the treated water transferred from the pressurized flotation tank;
an effluent filtration tank configured to remove and reduce floating total solids in the treated water transferred from the pre-fermentation tank; and
a conditioning tank configured to adjust and buffer a pH in the treated water transferred from the effluent filtration tank.

3. The green hydrogen generation apparatus of claim 1, wherein the MEC unit includes:
an MEC tank configured to generate hydrogen through microbial electrolysis using pre-treated soluble organic matter;
a storage tank configured to provide a flow rate from an external reaction tank to the MEC tank for supplying a circulating flow rate to the MEC tank; and
a trap tank configured to separate a gas and solution coming out through a negative electrode of the MEC tank and return the solution back to the storage tank.

4. The green hydrogen generation apparatus of claim 2, wherein the three-phase separator is configured to perform oil separation or solid-liquid separation by inputting a chemical cohesive agent set according to properties of the source water.

5. The green hydrogen generation apparatus of claim 2, wherein the pressurized flotation tank is configured to control a concentration of a particulate matter in the treated water transferred from the three-phase separator through a pressurized flotation process.

6. The green hydrogen generation apparatus of claim 2, wherein the pre-fermentation tank is configured to perform acid fermentation for the set retention time while adjusting the pH of the treated water.

7. The green hydrogen generation apparatus of claim 2, wherein the effluent filtration tank is provided with a cross-flow cross-filtration membrane to maintain the flow rate of the treated water at a set level.

8. The green hydrogen generation apparatus of claim 2, wherein the conditioning tank is configured to maintain the pH of the treated water at 6.0 to 7.5.

9. The green hydrogen generation apparatus of claim 1, wherein the post-treatment unit is configured to purify, compress, and liquefy the hydrogen gas generated through the MEC unit and transmit the hydrogen gas to a hydrogen charging station.

10. The green hydrogen generation apparatus of claim 1, wherein the post-treatment unit is connected to a wastewater treatment facility to remove organic components and nutrients generated through the MEC unit.
